(19) United States
(12) Patent Application Publication
(10) Pub. No.: US 2007/0239259 A1
(43) Pub. Date: Oct. 11, 2007

(54) Nitinol alloy design and composition for medical devices

(75) Inventor: John F. Boylan, Murrieta, CA (US)

Correspondence Address: FINNEGAN , HENDERSON , FARABOW, GARRETT& DUMMER;L.L.P / ABBOTT CARDIOVASCULAR SYSTEMS INC., 901 NEW YORK AVENUE , N.W, WASHINGTON, DC, 20001, US

(73) Assignee: Advanced Cardiovascular Systems Inc.

(21) Appl. No.: 11/723,339

(22) Filed: Mar. 19, 2007

Related U.S. Application Data
(63) Continuation of application No. 10/264,832, filed on Oct. 4, 2002, now abandoned.
Application No. 10/264,832 is a continuation-in-part of application No. 09/452,516, filed on Dec. 1, 1999.

(51) Int. Cl. A61F 2/06; B21D 51/10

(52) U.S. Cl. 623/1.15; 29/6.1; 606/194

(57) ABSTRACT

A stent and a delivery system for implanting the stent in a body lumen is disclosed. The stent is made from a superelastic alloy such as nickel-titanium or nitinol, and includes a ternary element in order to minimize the stress hysteresis of the superelastic material. The stress hysteresis is defined by the difference between the loading plateau stress and the unloading plateau stress of the superelastic material. The resulting delivery system has a small profile and includes a sheath covering the stent that has a thin wall. A guide wire core can also be made from the small stress hysteresis superelastic material.

### CROSS-REFERENCE TO RELATED APPLICATIONS

This is a continuation of co-pending application Ser. No. 10/264,832, filed Oct. 4, 2002, which is a continuation-in-part (CIP) of application Ser. No. 09/452,516, filed Dec. 1, 1999, both of which are incorporated herein by reference.

### BACKGROUND OF THE INVENTION

The present invention generally relates to medical devices including self-expanding endoprosthetic devices and guide wires. Regarding the former, self-expanding intraluminal vascular grafts, generally called stents, are adapted to be implanted in a body lumen, such as carotid arteries, coronary arteries, peripheral arteries, veins, or other vessels to maintain the patency of the lumen. These devices are frequently used in the treatment of atherosclerotic stenosis in blood vessels especially after percutaneous transluminal angioplasty (PTA) or percutaneous transluminal coronary angioplasty (PTCA) procedures, with the intent to reduce the likelihood of restenosis of a vessel. Stents are also used to support a body lumen, tack-up a flap or dissection in a vessel, or in general where the lumen is weak to add support. The present invention also relates to an intraluminal vascular graft that can be used in essentially any body lumen.

In expandable stents that are delivered with expandable catheters, such as balloon catheters, the stents are positioned over the balloon portion of the catheter and are expanded from a reduced diameter to an enlarged diameter greater than or equal to the inner diameter of the arterial wall, by inflating the balloon. Stents of this type can be expanded to an enlarged diameter by deforming the stent, by engagement of the stent walls with respect to one another, and by one way engagement of the stent walls together with endothelial growth onto and over the stent. Other stents are self-expanding, through the properties of the material constituting the stent or by design. Examples of intravascular stents can be found in U.S. Pat. No. 5,292,331 (Boneau); U.S. Pat. No. 4,580,568 (Gianturco); U.S. Pat. No. 4,856,516 (Hillstead); U.S. Pat. No. 5,092,877 (Pinchuk); and U.S. Pat. No. 5,514,154 (Lau et al.), which are incorporated herein by reference in their entirety.

The problems with some prior art stents, especially those of the expandable type, is that they are often stiff and inflexible. Often, the expandable type stents are formed from stainless steel alloys and the stents are constructed so that they are expanded beyond their elastic limit. Such stents are permanently deformed beyond their elastic limits and are capable of holding open a body lumen and maintaining patency of the body lumen. There are several commercially available stents that are widely used and generally implanted in the coronary arteries after a PTCA procedure.

One class of stents is implanted in vessels that are closer to the surface of the body, such as in the carotid arteries in the neck or in peripheral arteries and veins in the leg. Because these stents are so close to the surface of the body they are particularly vulnerable to impact forces that can partially or completely collapse the stent and thereby block fluid flow in the vessel. Since the prior art stents are plastically deformed, once collapsed or crushed they will remain so, permanently blocking the vessel. Thus, the prior art stents can pose an undesirable condition to the patient.

Other forces can impact the prior art stents and cause similar partial or total vessel blockage. Under certain conditions, muscle contractions might cause the prior art stents to partially or totally collapse and restrict blood flow in the vessel in which they are implanted.

Such important applications as mentioned above have prompted stent designers to use superelastic or shape memory alloys in their stent to exploit the materials' properties. An example of such shape memory alloy stents is disclosed in, for example, European Patent Application Publication No. EP0873734A2, entitled “Shape Memory Alloy Stent.” This publication suggests a stent for use in a lumen in a human or animal body having a generally tubular body formed from a shape memory alloy which has been treated so that it exhibits enhanced elastic properties. In particular, in the stress-strain curve exhibiting loading and unloading of the shape memory alloy material, the applicant suggests using a composition that results in a large difference between the loading and unloading curves, otherwise known as a wide hysteresis. The wide hysteresis means that the inward force required to compress the stent transversely once in place in the lumen is relatively high, while the outward force that the stent exerts on the lumen as it attempts to revert to its original undeformed configuration is relatively low. This can mean that the lumen will be resistant to being crushed by externally applied forces which can be a problem in the case of lumens close to the surface such as arteries in the thigh and neck. The publication further suggests use of specified ternary elements in a nickel titanium alloy to obtain a stent exhibiting a wider hysteresis in the stress-strain behavior in a loading and unloading cycle.

The evolution of superelastic and shape memory alloy stents progressed to use of ternary elements in combination with nickel-titanium alloys to obtain specific material properties. Use of a ternary element in a superelastic stent is shown in, for example, U.S. Pat. No. 5,907,893 to Zadno-Azizi et al. As a general proposition, there have been attempts at adding a ternary element to nickel-titanium alloys as disclosed in, for instance, U.S. Pat. No. 5,885,381 to Mitose et al.

On the other hand, the conventional efforts of using a ternary element in a superelastic material for a stent have focused only on a wider hysteresis in the stress-strain behavior in a loading or unloading cycle of the stent. Unfortunately, the greater the difference between the loading and unloading stress plateaus, the stronger the delivery system must be to accommodate any given level of stent performance. Typically, a stronger delivery system must also be larger and bulkier. This is a major drawback to conventional superelastic stents and delivery systems when the stent must be delivered through tortuous vessels at remote locations in the human anatomy.

In a typical PTCA procedure, a guiding catheter having a preformed distal tip is percutaneously introduced into the cardiovascular system of a patient in a conventional Seldinger technique and advanced therein until the distal tip is seated in the ostium of a desired coronary artery. A guide wire is positioned within an inner lumen of a dilatation catheter and then both are advanced through the guiding catheter to the distal end thereof. The guide wire is advanced out of the distal end of the guiding catheter into the patient's coronary vasculature until the distal end of the guide wire crosses a lesion to be dilated, then the dilatation catheter having an inflatable balloon on the distal portion thereof is advanced into the patient's coronary anatomy over the previously introduced guide wire until the balloon of the dilatation catheter is properly positioned across the lesion. Once in position across the lesion, the balloon is inflated to compress the arteriosclerotic plaque of the lesion against the inside of the artery wall. Through various other known procedures, a stent may be deployed and implanted at the lesion.

Conventional guide wires for angioplasty and other vascular procedures usually comprise an elongated core member with one or more tapered sections near the distal end and a flexible body such as a helical coil disposed about the distal portion of a core member. A shapeable member, which may be the distal extremity of the core member or a separate shaping ribbon that is secured to the distal extremity of the core member, extends through the flexible body and is secured to a rounded plug at the distal end of the flexible body. A torque applying mechanism is provided on the proximal end of the core member to rotate, and thereby steer, the guide wire while it is being advanced through the patient's vascular system.

A major requirement for guide wires and other guiding members, whether they be solid wire or tubular members, is that they have sufficient column strength to be pushed through a patient's vascular system or other body lumen without kinking. However, they must also be flexible enough to avoid damaging the blood vessel or other body lumen through which they are advanced. Efforts have been made to improve both the strength and flexibility of guide wires to make them more suitable for their intended uses, but these two properties are for the most part diametrically opposed to one another in that an increase in one usually involves a decrease in the other.

The prior art makes reference to the use of alloys such as nitinol (NiTi alloy) which have shape memory and/or superelastic characteristics in medical devices that are designed to be inserted into a patient's body. Because of these properties, nitinol has been employed in the fabrication of guide wires.

What has been needed and heretofore unavailable in the prior art is a superelastic stent and delivery system that applies a ternary element to the superelastic alloy in order to minimize the hysteresis. That hysteresis is defined by the difference between the loading and unloading plateau stresses of the material as plotted on a stress-strain curve. The present invention satisfies these needs. The present invention can further be applied to guide wires.

### SUMMARY OF THE INVENTION

The present invention is directed to a stent and a delivery system for implanting the stent in a body lumen, comprising a cylindrically-shaped stent including a superelastic alloy, wherein the alloy includes a ternary element, and wherein the alloy further includes a substantially small stress hysteresis; and a delivery system including a sheath having a distal end and a proximal end, wherein the stent is disposed inside the sheath at the distal end, and wherein the sheath has a small profile.

In a preferred embodiment, the superelastic alloy includes binary nickel-titanium alloys that exhibit superelasticity and have an unusual stress-strain relationship. More precisely, the superelastic curve is characterized by regions of nearly constant stress upon loading (referred to as the loading plateau stress) and unloading (unloading plateau stress). The loading plateau stress is always larger than the unloading plateau stress. The loading plateau represents the period during which martensite is being stress-induced in favor of the original austenitic structure. As the load is removed, the stress-induced martensite transforms back into austenite along the unloading plateau.

Self-expanding nitinol stents are collapsed (that is, loaded) and then constrained within a delivery system. At the point of delivery, the stent is released (that is, unloaded) and allowed to return to its original diameter. The stent is designed to perform various mechanical functions within the lumen, all of which are based upon the lower unloading plateau stress.

Importantly, the higher loading plateau stress therefore establishes the mechanical resistance the stent exerts against the delivery system. The greater the difference between these two plateaus is, the wider the hysteresis curve, and the stronger the delivery system must be to accommodate any given level of stent performance. The greater difference is described as a wide hysteresis. The conventional superelastic stent with a ternary element is designed to have a wider hysteresis resulting in a larger profile delivery system.

In the preferred embodiment of the present invention, however, an object is to decrease the stress hysteresis defined by the loading and unloading stress plateaus. This is accomplished by using a ternary element in addition to the superelastic alloy. As a result, the present invention stent and delivery system will enjoy an overall reduced delivery system profile for any given level of stent mechanical performance. Moreover, because of the smaller hysteresis and lower loading plateau stress for a given level of performance, the delivery system including the sheath can be made of a thinner wall material, leading to better flexibility.

As mentioned above, a preferred superelastic alloy is nickel-titanium or nitinol. In the exemplary embodiment, the ternary element may be palladium, platinum, chromium, iron, cobalt, vanadium, manganese, boron, copper, aluminum, tungsten, or zirconium.

The present invention is also directed to an elongated guide wire. The elongated guide wire comprises an elongated core having proximal and distal core sections, wherein the distal core section includes a superelastic alloy with a ternary element providing a substantially small stress hysteresis in the alloy. The proximal core section includes a high strength metal such as stainless steel. An optional torque-transmitting tube, made of a superelastic alloy, joins the proximal and distal core sections together. At least one flexible body such as a metallic helical coil is disposed about and secured to the distal core section. The guide wire core, including both the distal and proximal core sections, may optionally be made from one solid, uninterrupted section of material.

Other features and advantages of the present invention will become more apparent from the following detailed description of the invention when taken in conjunction with the accompanying exemplary drawings.

### BRIEF DESCRIPTION OF THE DRAWINGS

FIG. 1 is a partial cross-sectional view of a stent delivery system.

FIG. 2 shows, in a cross-sectional view, the stent delivery system of FIG. 1 with an optional expandable balloon.

FIG. 3 is a side elevational view, partially in section, depicting a stent mounted on a delivery catheter and expanded within a damaged vessel, pressing a damaged vessel lining against the vessel wall.

FIG. 4 is a side elevational view, partially in section, depicting an expanded stent within the vessel after withdrawal of the delivery catheter.

FIG. 5 is a plan view of the flattened strut pattern of an exemplary embodiment of a superelastic stent.

FIG. 6 is a typical stress-strain curve for a superelastic material.

FIG. 7 is a side elevational view of the guide wire embodying features of the present invention.

FIG. 8 is a side elevational view partially in section of an alternative embodiment guide wire having features of the present invention.

FIG. 9 is a cross-sectional view taken along line 9-9 of the guide wire shown in FIG. 8.

FIG. 10 is a cross-sectional view taken along line 10-10 of the guide wire shown in FIG. 8.

FIG. 11 is a side elevational view of an alternative embodiment guide wire having features of the present invention and employing a shaping ribbon.

### DETAILED DESCRIPTION OF THE PREFERRED EMBODIMENTS

During PTCA procedures it is common to use a dilation catheter to expand a diseased area to open the patient's lumen so that blood freely flows. Despite the beneficial aspects of PTCA procedures and its widespread and accepted use, it has several drawbacks, including the possible development of restenosis and perhaps acute thrombosis and sub-acute closure. This recurrent stenosis has been estimated to occur in seventeen to fifty percent of patients despite the initial PTCA procedure being successful. Restenosis is a complex and not fully understood biological response to injury of a vessel which results in chronic hyperplasia of the neointima. This neonintimal hyperplasia is activated by growth factors which are released in response to injury. Acute thrombosis is also a result of vascular injury and requires systemic antithrombotic drugs and possibly thrombolytics as well. This therapy can increase bleeding complications at the catheter insertion site and may result in a longer hospital stay. Sub-acute closure is a result of thrombosis, elastic recoil, and/or vessel dissection.

Several procedures have been developed to combat restenosis and sub-acute or abrupt closure, one of which is the delivery and implanting of an intravascular stent. Stents are widely used throughout the United States and in Europe and other countries. Generally speaking, the stents can take numerous forms, however, most common is a generally cylindrical hollow tube that holds open the vascular wall at the area that has been dilated by a dilation catheter. One highly regarded stent used and sold in the United States is sold under the tradename ACS Multi-Link Stent, which is made by Advanced Cardiovascular Systems, Inc., Santa Clara, Calif.

The stents of the present invention can have virtually any configuration that is compatible with the body lumen in which they are implanted. The stent should be configured so that there is a substantial amount of open area and preferably the open area to metal ratio is at least 80%. The stent also should be configured so that dissections or flaps in the body lumen wall are covered and tacked up by the stent.

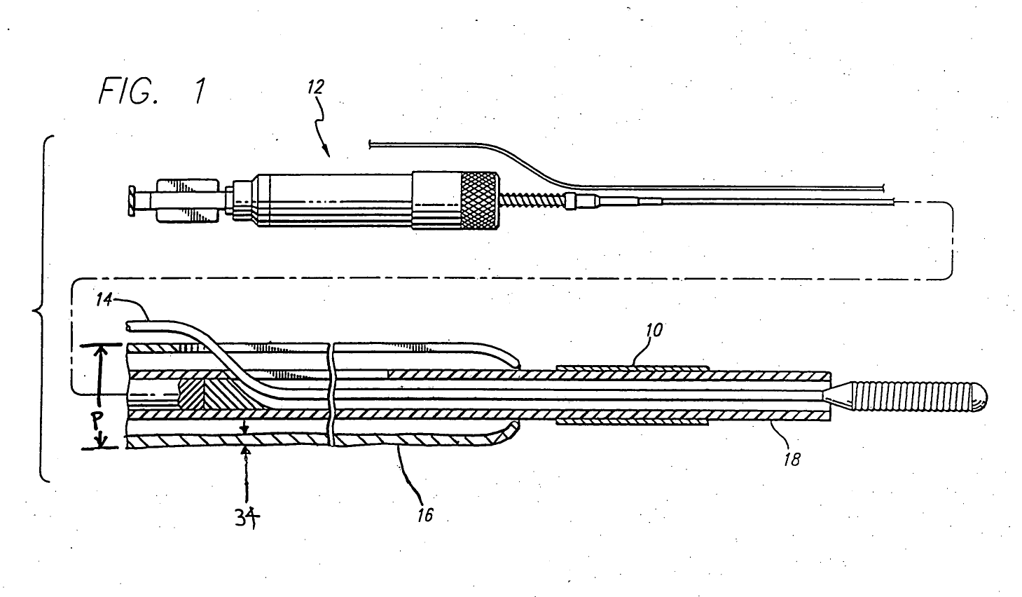

Referring to FIGS. 1 and 5, in a preferred embodiment, stent 10 of the present invention is formed partially or completely of alloys such as nitinol (NiTi) which have superelastic (SE) characteristics. Stent 10 is somewhat similar to the stent disclosed in U.S. Pat. No. 5,569,295, “Expandable Stents and Method for Making Same,” issued to Lam on Oct. 29, 1996, which is incorporated herein by reference in its entirety. Some differences of the present invention stent from that disclosed in the '295 patent is that the present invention stent is constructed of a superelastic material, and the strut pattern has changed. Of course, the configuration of stent 10 is just one example of many stent configurations that are contemplated by the present invention.

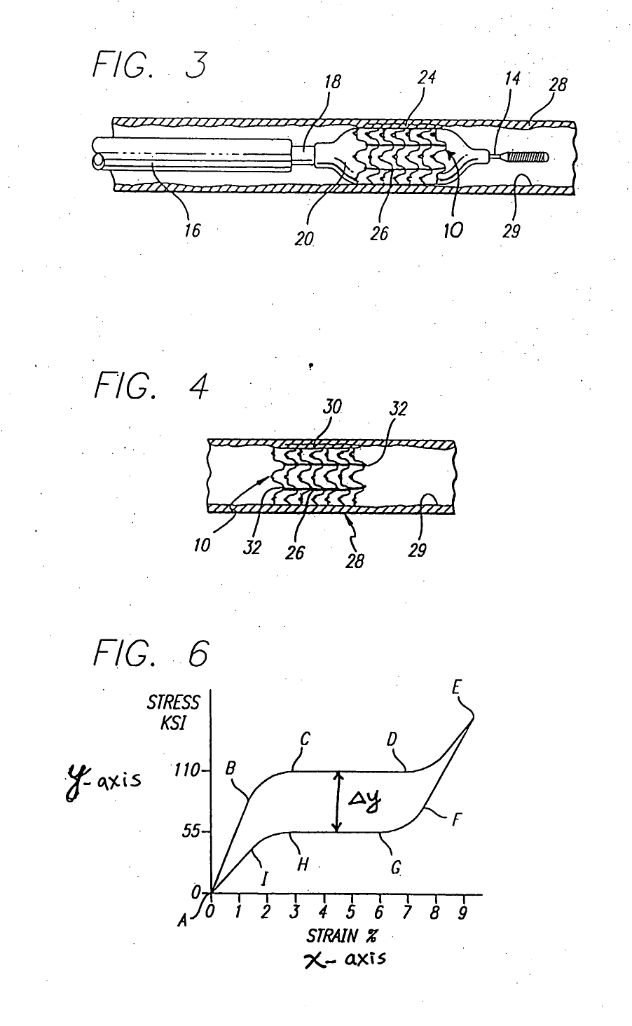

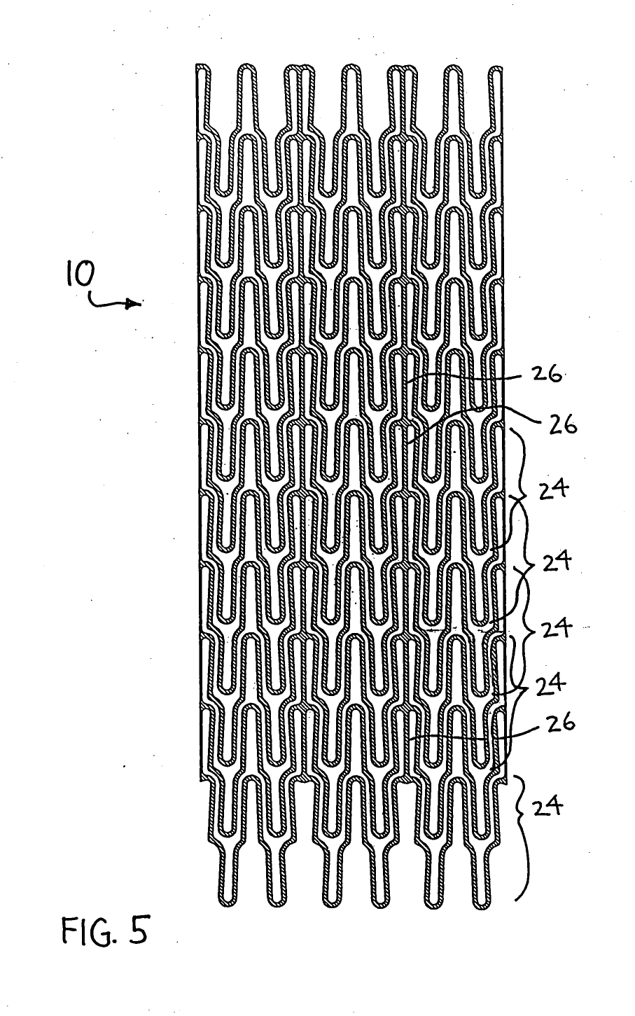

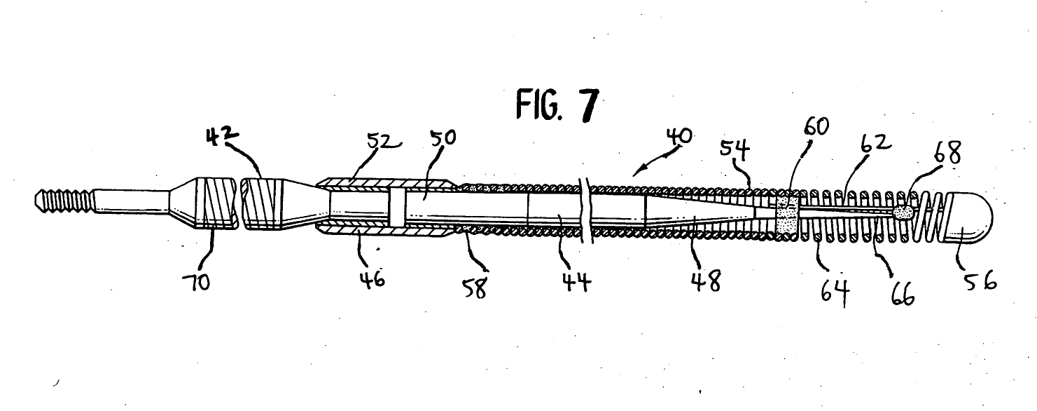

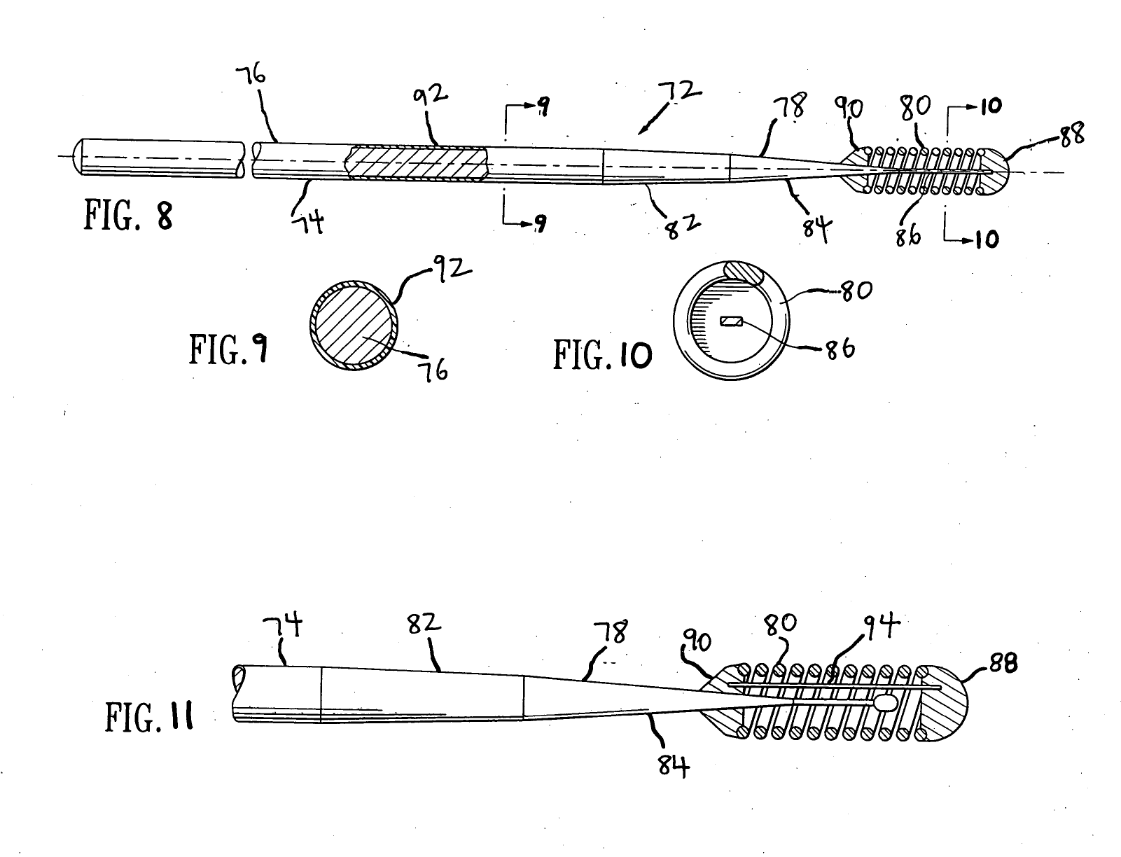

In keeping with the present invention, and turning to FIGS. 3, 4, and 5, stent 10 preferably includes a plurality of radially expandable cylindrical elements 24 disposed generally coaxially and interconnected at nodes 32 by interconnecting members 26 disposed between adjacent cylindrical elements 24. The deployed stent 10 is shown in FIG. 4 supporting an intimal flap 30 inside the diseased body lumen 29. The shape of the struts are designed so they can preferably be “nested.” This is best seen from the flattened plan view of FIG. 5. The serpentine shaped Struts are nested such that the extended portions of the struts of one cylindrical element 24 intrude into a complementary space within the circumference of an adjacent cylindrical element. In this manner, the plurality of cylindrical elements 24 can be more tightly packed lengthwise.

As introduced above, an exemplary stent of the present invention includes a superelastic material. The term “superelastic” refers to an isothermal transformation, more specifically stress inducing a martensitic from an austenitic phase. Alloys having superelastic properties generally have at least two phases: a martensitic phase, which has a relatively low tensile strength and which is stable at relatively low temperatures, and an austenitic phase, which has a relatively high tensile strength and which is stable at temperatures higher than the martensitic phase. Superelastic characteristics generally allow the metal stent to be deformed by collapsing and deforming the stent and creating stress which causes the NiTi to change to the martensitic phase. The stent is restrained in the deformed condition to facilitate the insertion into a patient's body, with such deformation causing the phase transformation. Once within the body lumen, the restraint on the stent is removed, thereby reducing the stress therein so that the superelastic stent can return to its original undeformed shape by the transformation back to the austenitic phase.

Returning to FIG. 1, the graphic illustrates, in a partial cross-sectional view, a rapid exchange stent delivery system that includes manipulating device 12, guide wire 14, delivery sheath 16, and intravascular catheter 18. This delivery system is just one example of a delivery system that may be used with the present invention. More details of this type of delivery system may be found in, for example, U.S. Pat. No. 5,458,615, “Stent Delivery System,” issued to Klemm et al. on Oct. 17, 1995, which is incorporated herein by reference in its entirety. Other delivery systems such as an over-the-wire delivery system may be used without departing from the scope of the instant invention.

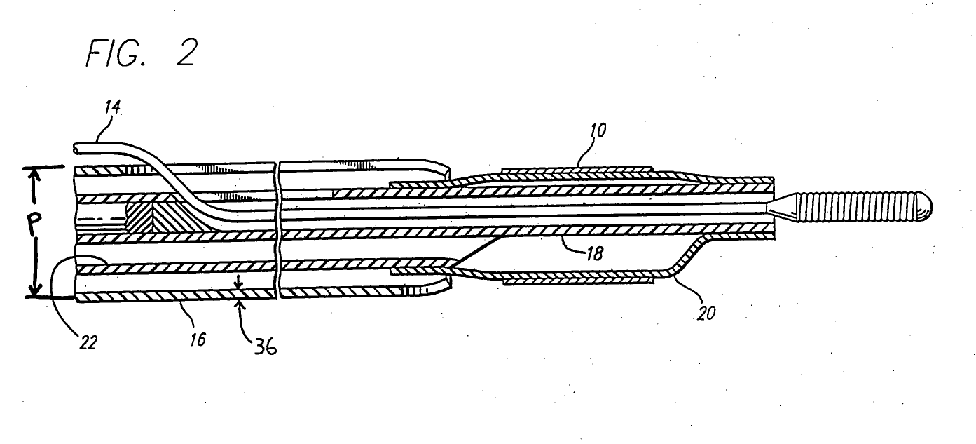

FIG. 2 depicts in a partial cross-sectional view a variation on the delivery system of FIG. 1, and includes optional expandable balloon 20 and optional balloon inflation lumen 22. Stent 10 is disposed over expandable balloon 20, and the entire assembly is kept underneath delivery sheath 16 until the moment stent 10 is deployed.

FIGS. 1 and 2 also depict delivery systems having a small delivery profile P. This reduced profile P is a beneficial attribute of the present invention stent and delivery system as a result of the stress-strain hysteresis curve of the superelastic material being minimized. This novel approach is described more fully below.

Stent 10 is preferably formed from a superelastic material such as NiTi and undergoes an isothermal transformation when stressed. The stent is first compressed to a delivery diameter, thereby creating stress in the NiTi alloy so that the NiTi is in a martensitic state having relatively low tensile strength. While still in the martensitic phase, the stent is mounted onto a catheter by known methods such as adhesives, or other restraining means. Alternatively, stent 10 can be mounted within delivery sheath 16 so that stent 10, which tends to spring back to a larger diameter, pushes radially outwardly against the inside diameter of sheath 16.

In its delivery diameter P, the overall diameter of the stent and catheter are less than the inside diameter of artery 28 or the vessel in which they are inserted. After stent 10 is inserted into the artery or other vessel, the stress exerted by stent 10 may be released by withdrawing delivery sheath 16 in a proximal direction, whereupon stent 10 immediately expands and returns to its original, undeformed shape by transforming back to the more stable austenitic phase. If expandable balloon 20 of FIG. 2 is implemented, stent 10 may be further expanded by inflation of expandable balloon 20 via balloon inflation lumen 22 by known methods.

FIG. 4 illustrates stent 10 in the expanded condition after the delivery system has been removed. If an external force is then applied to the artery, the stent temporarily at least partially collapses or deforms. As the stent deforms, stress in the NiTi alloy causes a phase transformation from the austenitic to the martensitic phase. When the external force is removed, the stress in stent 10 is removed so that the stent quickly transforms back from the martensitic phase to the austenitic phase. As this almost instantaneous transformation occurs, stent 10 returns to its fully expanded state and the artery remains open. When superelastic stent 10 is implanted in an artery, it maintains the patency of the artery while minimizing the risk of permanent arterial collapse at the implant site if the stent is temporarily deformed due to external forces. Thus, stent 10 imparts crush-resistant support at the implant site.

When stress is applied to a specimen of a metal such as nitinol exhibiting superelastic characteristics at a temperature at or above that which the transformation of the martensitic phase to the austenitic phase is complete, the specimen deforms elastically until it reaches a particular stress level where the alloy then undergoes a stress-induced phase transformation from the austenitic phase to the martensitic phase. As the phase transformation progresses, the alloy undergoes significant increases in strain with little or no corresponding increases in stress. The strain increases while the stress remains essentially constant until the transformation of the austenitic phase to the martensitic phase is complete. Thereafter, further increase in stress is necessary to cause further deformation. The martensitic metal first yields elastically upon the application of additional stress and then plastically with permanent residual deformation.

If the load on the specimen is removed before any permanent deformation has occurred, the martensite specimen will elastically recover and transform back to the austenitic phase. The reduction in stress first causes a decrease in strain. As stress reduction reaches the level at which the martensitic phase transforms back into the austenitic phase, the stress level in the specimen will remain essentially constant (but less than the constant stress level at which the austenitic crystalline structure transforms to the martensitic crystalline structure until the transformation back to the austenitic phase is complete); i.e., there is significant recovery in strain with only negligible corresponding stress reduction. After the transformation back to austenite is complete, further stress reduction results in elastic strain reduction. This ability to incur significant strain at relatively constant stress upon the application of a load and to recover from the deformation upon the removal of the load is commonly referred to as superelasticity.

The prior art makes reference to the use of metal alloys having superelastic characteristics in medical devices which are intended to be inserted or otherwise used within a patient's body. See, for example, U.S. Pat. No. 4,665,905 (Jervis) and U.S. Pat. No. 4,925,445 (Sakamoto et al.), which are incorporated by reference herein in their entirety.

FIG. 6 illustrates an example of a preferred stress-strain relationship of an alloy specimen, such as stent 10, having superelastic properties as would be exhibited upon tensile testing of the specimen. The relationship is plotted on x-y axes, with the x axis representing strain and the y axis representing stress. For ease of illustration, the x-y axes are labeled with typical pseudoelastic nitinol stress from 0 to 110 ksi and strain from 0 to 9 percent, respectively.

Looking at the plot itself in FIG. 6, the line from point A to point B represents the elastic deformation of the specimen. After point B the strain or deformation is no longer proportional to the applied stress and it is in the region between point B and point C that the stress-induced transformation of the austenitic phase to the martensitic phase begins to occur. There also can be an intermediate phase, called the rhombohedral phase, depending upon the composition of the alloy.

At point C moving toward point D, the material enters a region of relatively constant stress with significant deformation or strain. This constant or plateau region is known as the loading stress, since it represents the behavior of the material as it encounters continuous increasing strain. It is in this plateau region CD that the transformation from austenite to martensite occurs.

At point D the transformation to the martensitic phase due to the application of stress to the specimen is substantially complete. Beyond point D the martensitic phase begins to deform, elastically at first, but, beyond point E, the deformation is plastic or permanent.

When the stress applied to the superelastic metal is removed, the material behavior follows the curve from point E to point F. Within the E to F region, the martensite recovers its original shape, provided that there was no permanent deformation to the martensitic structure. At point F in the recovery process, the metal begins to transform from the stress-induced, unstable, martensitic phase back to the more stable austenitic phase.

In the region from point G to point H, which is also an essentially constant or plateau stress region, the phase transformation from martensite back to austenite takes place. This constant or plateau region GH is known as the unloading stress. The line from point I to the starting point A represents the elastic recovery of the metal to its original shape.

Binary nickel-titanium alloys that exhibit superelasticity have an unusual stress-strain relationship as just described and as plotted in the curve of FIG. 6. As emphasized above, the superelastic curve is characterized by regions of nearly constant stress upon loading, identified above as loading plateau stress CD and unloading plateau stress GH. Naturally, the loading plateau stress CD is always larger than the unloading plateau stress GH. The loading plateau stress represents the period during which martensite is being stress-induced in favor of the original austenitic crystalline structure. As the load is removed, the stress-induced martensite transforms back into austenite along the unloading plateau stress part of the curve. The difference in stress between the stress at loading CD and unloading stress GH defines the hysteresis of the system. This is identified as Δy of the curve in FIG. 6.

The present invention seeks to minimize the hysteresis of the superelastic material used to fabricate stent 10. Stent 10 is designed to perform various mechanical functions within a lumen, all of which are based upon the lower unloading plateau stress GH. Unloading plateau stress GH represents the behavior of the nitinol material when the stent is deployed.

On the other hand, the higher loading plateau stress CD establishes the mechanical resistance stent 10 exerts against the delivery system, and specifically delivery sheath 16. It represents the stress exerted by stent 10 when it is loaded into sheath 16. The greater the difference between the two plateaus CD and GH is (the hysteresis), the stronger the delivery system must be to accommodate any given level of stent performance. A stronger delivery system must necessarily be larger and bulkier, with a thicker delivery sheath 16.

Conversely, reducing the difference or Δy between the two plateaus CD and GH results in smaller hysteresis. The smaller the hysteresis is, the smaller and lower profile the delivery system has to be to accommodate any given level of stent performance. In one embodiment, the substantially small hysteresis is represented by a Δy of the curve that is less than about 40 ksi. Furthermore, the present invention delivery system can be smaller and constructed to a smaller profile due to the lower loading plateau stress CD, while maintaining a high hoop strength of the deployed, expanded stent represented by plateau stress GH.

In accordance with the present invention, stent 10 requires only a delivery system having a small delivery profile P as illustrated in the cross-sectional views of FIGS. 1 and 2. Furthermore, the wall thickness 34, 36 can be reduced as compared to a comparable performance stent not employing the present invention. Such a compact delivery system permits the physician better access and flexibility to reach tortuous arteries and vessels.

In sum, the present invention offers the potential to reduce overall delivery profile defined by loading stress CD for any given level of stent mechanical performance defined by unloading stress GH. In the present invention, this is accomplished by realizing the properties of superelastic nitinol, preferably in addition with a ternary element, as described in greater detail below.

The superelastic alloy of the present invention is preferably formed from a composition consisting essentially of about 30 to about 52 percent titanium and the balance nickel and up to 10 percent of one or more additional ternary alloying elements. Such ternary alloying elements may be selected from the group consisting of platinum, palladium, chromium, iron, cobalt, vanadium, manganese, boron, copper, aluminum, tungsten, or zirconium. In particular, the ternary element may optionally be up to 3 percent each of iron, cobalt, platinum, palladium, and chromium, and up to about 10 percent copper and vanadium. In one preferred embodiment of the present invention, the alloy has about 49.6 to about 49.8 percent of titanium, about 42.7 to about 42.9 percent of nickel, and about 7.5 percent of platinum, or alternatively one of the other ternary elements listed above. As used herein, all references to percent composition are atomic percent unless otherwise noted.

In another preferred embodiment, a NiTi stent with SME (shape memory effect) is heat-treated at approximately 500 degrees C. The stent is mechanically deformed into a first, smaller diameter for mounting on a catheter delivery system, such as the delivery system of FIG. 2, that includes expandable balloon 20 and balloon inflation lumen 22. After the stent has been expanded by the balloon and deployed against arterial wall 29 of artery 28, 45 degrees C. heat is applied causing the stent to return to its fully expanded larger diameter and be in contact with the arterial wall of the artery. The application of 45 degrees C. of heat is compatible with most applications in the human body, but it is not to be limited to this temperature as higher or lower temperatures are contemplated without departing from the invention. The 45 degrees C. temperature can be achieved in a conventional manner well known in the art such as by warm saline injected into the delivery catheter and balloon.

The shape memory characteristics allow the devices to be deformed to facilitate their insertion into a body lumen or cavity and then to be heated within the body so that the device returns to its original shape. Again, alloys having shape memory characteristics generally have at least two phases: a martensitic phase, which has a relatively low tensile strength and which is stable at relatively low temperatures, and an austenitic phase, which has a relatively high tensile strength and which is stable at temperatures higher than the martensitic phase.

Shape memory characteristics are imparted to the alloy by heating the metal to a temperature above which the transformation from the martensitic phase to the austenitic phase is complete; i.e., a temperature above which the austenitic phase is stable. The shape of the metal during this heat treatment is the shape “remembered.” The heat-treated metal is cooled to a temperature at which the martensitic phase is stable, causing the austenitic phase to transform to the martensitic phase. The metal in the martensitic phase is then plastically deformed, e.g., to facilitate the entry thereof into a patient's body. Subsequent heating of the deformed martensitic phase to a temperature above the martensite to austenite transformation temperature causes the deformed martensitic phase to transform to the austenitic phase. During this phase transformation the metal reverts back to its original shape.

The recovery or transition temperature may be altered by making minor variations in the composition of the metal and in processing the material. In developing the correct composition, biological temperature compatibility must be determined in order to select the correct transition temperature. In other words, when the stent is heated, it must not be so hot that it is incompatible with the surrounding body tissue. Other shape memory materials may also be utilized, such as, but not limited to, irradiated memory polymers such as autocrosslinkable high density polyethylene (HDPEX).

Shape memory alloys are known in the art and are discussed in, for example, “Shape Memory Alloys,” Scientific American, Vol. 281, pp. 74-82 (November 1979), incorporated herein by reference.

Shape memory alloys undergo a transition between an austenitic state and a martinsitic state at certain temperatures. When they are deformed while in the martinsitic state they will retain this deformation as long as they are retained in this state, but will revert to their original configuration when they are heated to a transition temperature, at which time they transform to their austenitic state. The temperatures at which these transitions occur are affected by the nature of the alloy and the condition of the material. Nickel-titanium based alloys (NiTi), wherein the transition temperature is slightly lower than body temperature, are preferred for the present invention. It is desirable to have the transition temperature set at just below body temperature to insure a rapid transition from the martinsitic state to the austenitic state when the stent is implanted in a body lumen.

Turning again to FIG. 3, stent 10 is formed from a shape memory alloy, such as NiTi discussed above. After stent 10 is inserted into artery 28 or other vessel, expandable balloon 20 is inflated via balloon inflation lumen 22 by conventional means such that the stent is expanded radially outwardly. The stent then immediately expands due to contact with the higher temperature within artery 28 as described for devices made from shape memory alloys. Again, if an external force is then applied to the artery, stent 10 temporarily at least partially collapses. But stent 10 then quickly regains its former expanded shape due to its shape memory qualities. Thus, the crush-resistant stent, having shape memory characteristics, is implanted in a vessel, thereby maintaining the patency of a vessel while minimizing both the risk of permanent vessel collapse and the risk of dislodgment of the stent from the implant site if the stent is temporarily deformed due to external forces.

The present invention is applicable to guide wires. FIG. 7 is a side elevational view partially in section of an exemplary embodiment guide wire 40 employing features of the present invention. The guide wire 40 comprises an elongated, relatively high strength proximal portion 42, a relatively short distal portion 44 which is formed substantially of a superelastic alloy material and a connector element 46 formed substantially of a superelastic alloy material and which connects the proximal end of the distal portion 44 to the distal end of the proximal portion in a torque transmitting relationship. The distal portion 44 has at least one tapered section 48 that becomes smaller in the distal direction. The connector element 46 is preferably a hollow, tubular shaped structure having an inner lumen extending therethrough which is adapted to receive the proximal end 50 of the distal portion 44 and the distal end 52 of the proximal portion 42. The ends 50, 52 may be press fit into the connector element 46, or they may be secured therein by crimping, swaging the connector, or by means such as a suitable adhesive, weld, braze, or solder.

A helical coil 54 is disposed about the distal portion 44 and has a rounded plug 56 at the distal end thereof. The helical coil 54 is preferably secured to the distal portion 44 at proximal location 58 and at intermediate location 60 by solder and by the distal end thereof to the rounded plug 56, which is usually formed by soldering or welding the distal end of the helical coil 54 to the distal tip of the optional shaping ribbon 62. Preferably, the most distal section 64 of the helical coil 54 is made of a radiopaque metal such as platinum, or platinum-nickel alloys to facilitate the identification thereof while it is disposed within a patient's body under a fluoroscope or x-ray. The most distal section 64 is preferably stretched about 10% to about 30% of the un-stretched length of the helical coil 54.

A most distal part 66 of the distal portion 44 is optionally flattened into a rectangular or square shaped cross-section and preferably provided with a rounded tip 68. The rounded tip 68 may be a bead of solder used to minimize the inadvertent passage of the most distal part 66 through the spacing between the stretched distal section 64 of the helical coil 54.

The exposed portion of the elongated proximal portion 42 may be covered with a coating 70 of lubricious material such as polytetrafluroroethylene (sold under the trademark TEFLON) or other suitable lubricious coatings such as polysiloxane.

The elongated proximal portion 42 of the guide wire 40 is generally about 130 to about 140 cm in length with an outer diameter of about 0.006 to about 0.018 inch for coronary use. Large diameter guide wires may be employed in peripheral arteries and other body lumens. The lengths of the smaller diameter and tapered sections can range from about 2 to about 20 cm, depending upon the stiffness or flexibility desired in the final product. The helical coil 54 is about 20 to about 45 cm in length, has an outer diameter about the same size as the diameter of the elongated proximal portion 42, and is made from wire about 0.002 to 0.003 inch in diameter. More than one helical coil can be used. The shaping ribbon and the flattened most distal part 66 of the distal portion 44 may have rectangular cross-sections which usually have dimensions of about 0.001 by 0.003 inch.

The present invention guide wire 40 includes a superelastic nitinol distal portion 44 and optionally the connector element 46. Both are preferably made in accordance with the nickel-titanium and ternary element compositions described above. The connector element 46 may be a binary nitinol in one alternative embodiment.

A presently preferred method for making the final configuration of the superelastic portions of the guide wire 40 is to cold work, preferably by drawing, a rod or tubular member having a composition according to the relative proportions described above, and then heat treating the cold worked part while it is under stress to impart a shape memory thereto. Typical initial transverse dimensions of the rod or the tubular member are about 0.045 inch and about 0.25 inch respectively. If the final product is to be tubular, a small diameter ingot, e.g. 0.25 to about 1.5 inch in diameter and 5 to about 30 inches in length, may be formed into a hollow tube by extruding or by machining a longitudinal center hole therethrough and grinding the outer surface thereof smooth. Before drawing the solid rod or tubular member, it is optionally annealed at a temperature of about 500° C. to about 750° C., typically about 650° C., for about 30 minutes in a protective atmosphere such as argon to relieve essentially all internal stresses. In this manner, all of the specimens start the subsequent thermal mechanical processing in essentially the same metallurgical condition so that products with consistent final properties are obtained. Such treatment also provides the requisite ductility for effective cold working.

The stress-relieved stock is cold worked by drawing to effect a reduction in the cross-sectional area thereof of about 30% to 70%. The metal is drawn through one or more dies or appropriate inner diameter with a reduction per pass of about 10% to 50%. Other forms of cold working can be employed such as swaging.

Following cold work, the drawn wire or hollow tubular product is heat treated at a temperature between 350° C. and about 600° C. for about 0.5 to about 60 minutes. Preferably, the drawn wire or hollow tubular product is simultaneously subjected to a longitudinal stress between about 5% and about 50%, preferably about 10% to about 30% of the tensile strength of the material (as measured at room temperature) in order to impart a straight memory shape to the metal and to insure that any residual stresses therein are uniform. This memory-imparting heat treatment also fixes the austenite-martensite transformation temperature for the cold worked metal. By developing a straight memory and maintaining uniform residual stresses in the superelastic material, there is little or no tendency for a guide wire made of this material to whip when it is torqued within a patient's blood vessel. Other processing details for guide wires can be found in, for example, U.S. Pat. No. 5,695,111 to Nanis, et al., or U.S. Pat. No. 5,341,818 to Abrams, et al., whose contents are hereby incorporated by reference.

The nitinol hypotube from which the connector element 46 is formed generally may have an outer diameter from about 0.006 inch to about 0.002 inch with wall thicknesses of about 0.001 to about 0.004 inch. A presently preferred superelastic hypotube for the connector element 46 has an outer diameter of about 0.014 inch and a wall thickness of about 0.002 inch.

FIG. 8 is a side elevational view partially in section of an alternative embodiment guide wire 72. The guide wire 72 of this exemplary embodiment has a core member 74 with a proximal core section 76, a distal core section 78, and a helical coil 80. The distal core section 78 has a first tapered segment 82 and a second tapered core segment 84 which is distally contiguous to the first tapered core segment 82. The second tapered segment 84 tapers at a greater degree than the first tapered segment 82 and this additional taper provides a much smoother transition when the distal portion of the guide wire 72 is advanced through a tortuous passageway of a patient.

A flattened distal tip 86 extends from the distal end of the second tapered core segment 84 to the body of solder 88 that secures the flattened distal tip 86 of the core member 74 to the distal end of the helical coil 80. A body of solder 90 secures the proximal end of the helical coil 80 to an intermediate location on the second tapered segment 84. As seen in the embodiment of FIG. 8, the core member 74 optionally extends, continuously and uninterrupted, from the proximal end to the distal end of the guide wire 72.

The core member 74 is preferably coated with a lubricious coating 92 such as a fluoropolymer, e.g. TEFLON available from DuPont, which extends the length of the proximal core section 76. The distal core section 78 is also covered with a lubricious coating (not shown) such as a MICROGLIDE coating used by Advanced Cardiovascular Systems, Inc. Hydrophilic coatings may also be employed.

FIG. 11 is a partial side elevational view of the distal core section of an alternative embodiment guidewire that has a separate shaping ribbon extending from the distal extremity of the core member 74 to the distal end of the helical coil 80. In this alternative embodiment, the flattened distal tip 86 of the core member 74 shown in FIG. 8 is replaced with a shaping ribbon 94 which is secured by its distal end to the distal end of the coil 80 and by its proximal end to the distal extremity of the core member 74.

In the embodiments illustrated in FIG. 8 and FIG. 11, the guide wires may employ the superelastic nickel-titanium alloys with a ternary element as described above. It is also possible to use non-superelastic metals such as steels for the core member. For example, the entire core member of the guide wire may be made from high tensile stainless steel, or high-ten 304 stainless steel. Other high strength metals, some of which are precipitation hardenable, include 304 stainless steel, MP35N, and L605.

The tapered segments 82, 84 shown in FIGS. 8 and 11 may be fashioned to create a linear change in stiffness. This is disclosed in, for example, U.S. Pat. No. 6,390,993 to Cornish, et al., whose contents are hereby incorporated by reference herein.

While the present invention has been illustrated and described herein in terms of, for example, a guide wire or a superelastic stent and delivery system wherein the stent employs a ternary element to minimize the hysteresis defined by the difference in the loading plateau stress and the unloading plateau stress of the superelastic material, it is apparent to those skilled in the art that the present invention can be used in other instances. Other modifications and improvements may be made without departing from the scope of the present invention.

1-21. (canceled)

22. A stent for use in a body lumen, comprising:
a superelastic alloy, wherein the superelastic alloy comprises at least one ternary element in an amount effective to decrease the stress hysteresis of the superelastic alloy by lowering the loading plateau stress below that of the superelastic alloy without a ternary element without changing the unloading plateau stress.

23. An elongated guide wire, comprising:
an elongated core having proximal and distal core sections;
wherein the distal core section comprises a superelastic alloy comprising at least one ternary element in an amount effective to decrease the stress hysteresis of the superelastic alloy by lowering the loading plateau stress below that of the superelastic alloy without a ternary element without changing the unloading plateau stress; and
a flexible body disposed about and secured to the distal core section.

24. A method for providing an elongated guide wire, comprising:
providing an elongated core having proximal and distal core sections;
forming the distal core section from a superelastic alloy that comprises at least one ternary element in an amount effective to decrease the stress hysteresis of the superelastic alloy by lowering the loading plateau stress below that of the superelastic alloy without a ternary element without changing the unloading plateau stress; and
securing a flexible body disposed about the distal core section.
